(19) United States
(12) Patent Application Publication
(10) Pub. No.: US 2009/0156921 A1
(43) Pub. Date: Jun. 18, 2009

(54) CARDIAC ABLATION CATHETER WITH OXYGEN SATURATION SENSOR

(76) Inventor: Huisun Wang, Maple Grove, MN (US)

Correspondence Address: SJM/AFD - DYKEMA;c/o CPA Global, P.O. Box 52050, Minneapolis, MN, 55402, US

(21) Appl. No.: 11/959,214

(22) Filed: Dec. 18, 2007

(51) Int. Cl. A61B 5/145; A61B 18/14

(52) U.S. Cl. 600/364; 606/41

(57) ABSTRACT

Positioning of the distal end of a catheter at a desired location, for example, in the desired atrium of a patient's heart, can be determined or verified through measurement of blood gas values proximate to the distal end. In one embodiment, an oximeter is used to monitor oxygen saturation, for example, to distinguish between de-oxygenated venous blood and well-oxygenated arterial blood. Optical signals may be transmitted to the distal end of the catheter and received therefrom via optical fibers. Specifically, a catheter (300) has a number of optical fiber ends (308, 310 and 312) disposed at a distal end (304) thereof. A first fiber end (310) transmits the red and infrared optical signals. Fiber ends (308 and 312) are used for detecting reflected optical signals. The optical signals are then processed by a detector and an oximeter instrument to provide oxygen saturation readings that can indicate the position of the distal end of the catheter within the patient's heart or successful penetration of the interatrial septum.

### BACKGROUND OF THE INVENTION

a. Field of the Invention

The present invention relates generally to catheter positioning and, in particular to determining or verifying that a catheter is positioned at a desired location, e.g., in the desired atrium of the heart, based on blood gas measurements (for example, oxygen saturation, carbon dioxide concentration or the like) of the blood or perfused tissue. The invention is also useful for in vivo blood gas measurements independent of any guidance objective.

b. Background Art

Catheters have been in use for medical procedures for many years. Catheters can be used for medical procedures to examine, diagnose, and treat while positioned at a specific location within the body that is otherwise inaccessible without more invasive procedures. During these procedures a catheter is typically inserted into a vessel near the surface of the body and is guided to a specific location within the body for examination, diagnosis, and treatment. For example, catheters can be used to convey an electrical stimulus to a selected location within the human body, e.g., for tissue ablation. Catheters with sensing electrodes can be used to monitor various forms of electrical activity in the human body, e.g., for electrical mapping.

Catheters are used increasingly for medical procedures involving the human heart. Typically, the catheter is inserted in an artery or vein in the leg, neck, or arm of the patient and threaded, sometimes with the aid of a guide wire or introducer, through the vessels until a distal tip of the catheter reaches the desired location for the medical procedure in the heart. In the normal heart, contraction and relaxation of the heart muscle (myocardium) takes place in an organized fashion as electrochemical signals pass sequentially through the myocardium.

Sometimes abnormal rhythms occur in the heart, which are referred to generally as arrhythmia. The cause of such arrhythmia is generally believed to be the existence of an anomalous conduction pathway or pathways that bypass the normal conduction system. These pathways can be located in the fibrous tissue that connects the atrium and the ventricle.

An increasingly common medical procedure for the treatment of certain types of cardiac arrhythmia is catheter ablation. During conventional catheter ablation procedures, an energy source is placed in contact with cardiac tissue (e.g., associated with an anomalous conduction pathway) to create a permanent scar or lesion that is electrically inactive or noncontractile. The lesion partially or completely blocks the stray electrical signals to lessen or eliminate arrhythmia.

Ablation of a specific location within the heart requires the precise placement of the ablation catheter within the heart. Precise positioning of the ablation catheter is especially difficult because of the physiology of the heart, particularly because the heart continues to beat throughout the ablation procedures. Commonly, the placement of the catheter is guided by fluoroscopy sometimes using a contrast agent and/or by a combination of electrophysiological guidance and computer generated maps/models that may be generated during a mapping procedure. Additionally, in some cases, ultrasonic guidance is provided by introducing an ultrasound transducer to the procedure site via a separate catheter. Even with these guidance techniques, proper positioning of the distal end of the catheter for certain procedures may still involve considerable uncertainty. Moreover, these guidance techniques may complicate the procedure or expose the patient to increased risk, additional procedures or inconvenience.

### BRIEF SUMMARY OF THE INVENTION

The present inventor has recognized that positioning of the distal end of a catheter at a desired location can be determined or verified through measurement of blood gas values proximate to the distal end. In particular, it is expected that venous blood will have a lower oxygen saturation (and higher carbon dioxide concentration) than arterial blood. Accordingly, blood gas measurements or changes therein may be useful to indicate that the distal end of the catheter is positioned in a vein or the right side of the heart (e.g., the right atrium), on the one hand, or in an artery or the left side of the heart (e.g., in the left atrium), on the other. This information may be useful in certain catheter guidance applications.

The case of the transeptal procedures is illustrative. Access to the left atrium and pulmonary veins often requires performing a transeptal procedure where a catheter or other instrument is pushed through the interatrial septum between the left and right atriums. Such an instrument preferably punctures the septum at its thinnest location, for example, the fossa ovalis. This location is not readily determined using conventional imaging techniques such as fluoroscopy or intracardial mapping. Instead, the physician determines the puncture location based on his/her experience using the electrode catheter to probe the interatrial septum to identify the most compliant location, typically the fossa ovalis. Such experience only comes with time, and may be quickly lost if the physician does not perform the procedure on a regular basis.

It will thus be appreciated that confirmation that the interatrial septum has been penetrated and that the distal end of the catheter is in the desired atrium may be useful to a physician in this example, passage of the distal end of the catheter from one atrium to the other by traversing the interatrial septum will generally be accompanied by a transition from contact with deoxygenated venous blood to well oxygenated arterial blood or vice-versa. Accordingly a blood gas measurement, e.g., an in vivo measurement, or a monitored change in an associated value, can indicate that the distal end of the catheter is positioned in the correct atrium for the procedure under consideration.

In addition, such in vivo measurements may be useful in monitoring a patient independent of catheter guidance functionality. Indeed, such in vivo measurements may be more reliable than conventional pulse oximetry measurements which attempt to distinguish effects due to arterial blood from effects associated with other absorbers/attenuators, and that can be difficult in cases of patient motion and low perfusion.

Blood gas measurements can be made, for example, using optical or chemical processes, and any appropriate measurement can be employed in the context of the present invention. By way of example, oxygen saturation can be measured optically. In particular, oxygenated blood has different light transmission or absorption characteristics than deoxygenated blood. This is reflected in the observation that well-oxygenated arterial blood appears bright and red whereas deoxygenated or venous blood appears dark and bluish. Optical techniques that provide an indication of color or color change may therefore be used to measure oxygen saturation in vivo, to determine catheter position and/or to guide a catheter as discussed above.

Conventional oximeters typically utilize optical sources (e.g., LEDs) of two or more wavelengths. The sources are used to illuminate perfused tissue. The resulting optical signals are detected after they have been transmitted through or reflected from the perfused tissue. In either case, the optical signals are attenuated due to interaction with the patient's blood/perfused tissue. In these applications, the ratio of an attenuation related value for the red signal to a similar value for the infrared signal can be used to compute oxygen saturation.,

It may be expedient to use conventional oximetry processing in this regard and the resulting values are useful for patient monitoring. However, simplified processes may be adequate for the noted objective of catheter positioning. In particular, it is expected that oxygen saturation in the left atrium will be very high, generally above 95% and often at least about 99%. On the other hand, oxygen saturation in the left atrium will be considerably lower, generally below 90% and often below about 80%. Accordingly, high accuracy is not necessary to distinguish between the atria.

Moreover, an at least partially catheter-borne instrument can directly access the patient's blood substantially without interference associated with other optical attenuators. Accordingly, various processing associated with addressing variations in optical signal wavelengths, certain conventional pulse oximetry signal-to-noise ratio, addressing patient motion and the like may be unnecessary. Indeed, the conventional use of multiple optical sources at specific red and infrared wavelengths may be unnecessary. However, as noted above, the use of conventional instrumentation and processing may be expedient and provides information useful for patient monitoring.

Thus, in accordance with one aspect of the present invention, a method is provided for positioning a catheter at a desired location in a patient's body. The method includes the steps of inserting the catheter into the patient's body, performing at least one blood gas measurement in relation to the catheter and using the at least one blood gas measurement to assist in positioning the catheter at the desired location in the patient's body. For example, the blood gas measurement may involve measuring an oxygen saturation value, a carbon dioxide concentration value or other value effective to distinguish arterial blood from venous blood. Such values may be measured optically, chemically or by any other suitable process.

In one implementation, oximetry structure is disposed at or near the distal end of the catheter. For example, one or more LEDs, for transmitting optical signals, and a detector may be disposed on the catheter. Alternatively, optical fibers may be used to transmit the optical signals to the distal end of the catheter and/or to receive the optical signals. In this manner, the optical signals can be used to make blood gas measurements. The blood gas measurements may then be used to determine or verify that the distal end of the catheter is positioned at the desired location for a medical procedure. For example, an oxygen saturation value may be compared to a threshold(s) to distinguish between arterial blood and venous blood, or a change in oxygen saturation may be used to identify a transition between arterial and venous blood. This information can be used to ensure proper positioning for a medical procedure such as cardiac ablation. In this regard, instrumentation for such a procedure, such as an ablation electrode, may be disposed on the same catheter as the blood gas measurement instrumentation.

In accordance with another aspect of the present invention, a catheter apparatus is provided. The catheter apparatus includes a catheter having a distal end for introduction into a patient to a desired location via a blood vessel of the patient, blood gas measurement structure proximate to the distal end of the catheter, and medical procedure structure disposed proximate to the distal end of the catheter. The blood gas measurement structure may include structure for chemically or optically measuring a blood gas value such as oxygen saturation or carbon dioxide concentration. In one embodiment, oximetry structure, as discussed above, is disposed adjacent to a distal end of the catheter. The medical procedure structure may include a diagnostic or therapeutic electrode such as a cardiac ablation electrode.

### BRIEF DESCRIPTION OF THE DRAWINGS

FIG. 1 is a schematic view of a catheter system incorporating an oximeter in accordance with the present invention.

FIG. 2 is a side cross-sectional view of a distal end portion of a catheter including oximeter structure in accordance with one embodiment of the present invention.

FIG. 3 is a side cross-sectional view of a distal end portion of a catheter in accordance with another embodiment the present invention.

FIG. 4 is a flow chart illustrating a process for performing a medical procedure using a catheter with oximetry structure in accordance with the present invention.

FIG. 5 is a side cross-sectional view of a distal end portion of a catheter in accordance with a still further embodiment of the present invention.

### DETAILED DESCRIPTION OF THE INVENTION

The present invention relates to certain structure and methodology for using blood gas measurements to assist in positioning a catheter for a medical procedure. A variety of blood gas measurements may be performed in this regard, including oxygen saturation measurements, carbon dioxide concentration measurements or other blood gas measurements, and these measurements may be performed optically, chemically or in any other appropriate manner. In addition, a variety of types of medical procedures may be assisted in this regard including, for example, diagnostic and therapeutic procedures. In the following description, the invention is set forth in the context of an ablation catheter including oximetry structure for obtaining oxygen saturation measurements. Moreover, the invention is described with respect to specific procedures including transseptal procedures. While this structure and these applications represent an advantageous context for application of the present invention, it will be appreciated that the invention is not limited to such structure and applications. Accordingly, the following description should be understood as providing an exemplary discussion of the invention and not by way of limitation.

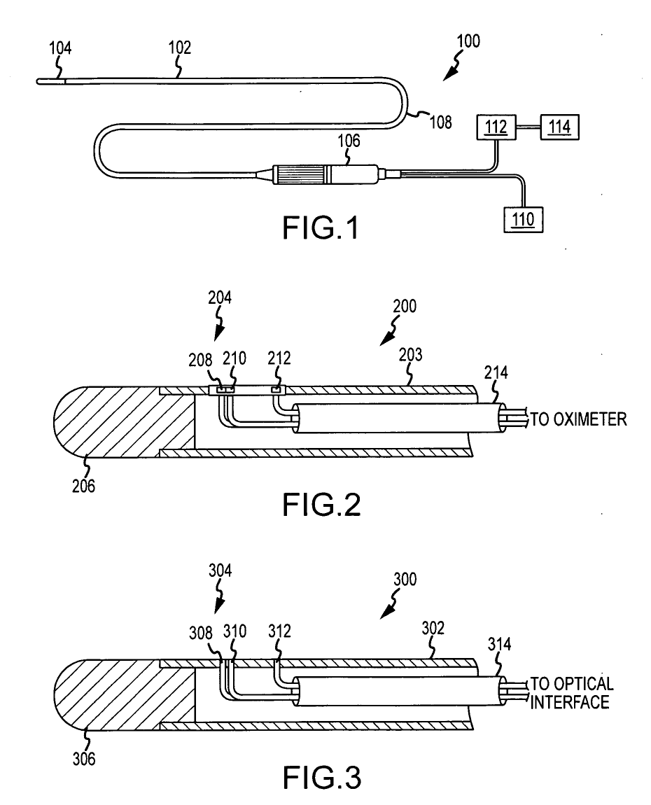

FIG. 1 illustrates a catheter system 100 in accordance with the present invention. The catheter system 100 includes a catheter 102 having a distal end 104 for introduction into a patient to a desired location. For example, the catheter may be introduced into the patient through an artery or vein, for example, in the patient's neck, arm or leg, and then threaded through the vessel to the patient's heart. As discussed above, for transseptal procedures, the distal end 104 of the catheter 102 penetrates the interatrial septum to gain access to the desired location for a medical procedure such as an ablation procedure to correct cardiac arrhythmia.

One application of the present invention is to provide an indication to a physician that the distal end 104 of the catheter 102 is positioned either in the left atrium or the right atrium. This is accomplished by obtaining blood gas measurements that are readily used to distinguish between the deoxygenated venous blood of the right side of the heart, including the right atrium, from the well-oxygenated arterial blood of the left side of the heart, including the left atrium.

In the embodiments described below, optical oximetry measurements are used in this regard. Such measurements may measure oxygen saturation or carbon dioxide concentration. Moreover, these measurements may be simple color or attenuation measurements or may be pulsatile waveform or photoplethysmographic measurements. In the implementations described below, an oximeter is used to make conventional photoplethysmographic measurements so as to determine oxygen saturation.

The oxygen saturation of arterial blood, or SₐO₂, is readily distinguished from the oxygen saturation of venous blood, SᵥO₂, particularly where such measurements are performed in the left and right atria. In particular, for healthy patients, it is expected that the measured value of SₐO₂ will generally be in excess of 95% and often about 99% or more. By contrast, the measured value of SᵥO₂ is expected to be below 90% and often below about 80%. Accordingly, any appropriate observations can be used to indicate the position of the distal end of the catheter or the transition between the atria including threshold comparisons or changes in measured oxygen saturation.

Thus, for example, a physician may monitor oxygen saturation readings during a transseptal procedure to identify a change in oxygen saturation indicating a transition from arterial blood to venous blood or vice versa. For example, a change in measured oxygen saturation of at least 5% and, more preferably, at least 10% may indicate a transition between the atria. Additionally or alternatively, a physician may use an oxygen saturation measurement to confirm the position of the distal end of the catheter that has been preliminarily determined by the physician based on an imaging system or tactile feedback indicating that the interatrial septum has been penetrated. For example, the physician may base this determination on a comparison to a threshold of, for example, 90% oxygen saturation or some other value including a patient-dependent value.

This oxygen saturation monitoring process may also be at least partially automated. In this regard, the oximeter instrument may execute algorithms to identity specified conditions. For example, the physician or other person involved in the medical procedure may use a user interface to identify a procedure to be performed, e.g., a transseptal procedure, and request notification when the distal end of the catheter has reached the desired location The physician may define thresholds to be utilized for making this determination or default thresholds may be defined in the processing logic. In either case, the logic may monitor oxygen saturation readings to identify an appropriate condition, e.g., transition of the oxygen saturation readings from below 90% (or other threshold) to above 90%, or a change in the monitored oxygen saturation value of at least 5% or at least 10%. Averaging filters may be used in this regard to distinguish between transient changes, for example, triggered by patient motion or other artifact, and persistent changes that more likely indicate passage of the distal end of the catheter across the interatrial septum. Whatever the condition is that is defined by the logic, when the condition is satisfied, an indication may be provided to the physician in any appropriate way. For example, an audio or visual output may be provided by the oximeter instrument or a vibration device in the catheter handle may be triggered to provide a tactile indication to the physician.

Referring again to FIG. 1, the illustrated catheter system 100 further includes a handle 106 that is used by the physician to manipulate the catheter 102 The system 100 also includes an RF generator 110 for generating an electrical signal that is transmitted to an electrode at the distal end 104 of the catheter 102 to execute the desired procedure such as cardiac ablation. An oximeter instrument 114 receives electrical signals representative of optical signals received via the catheter 102 so as to perform the oxygen saturation calculations and execute other logic, as noted above. Depending on the implementation, as will be better understood from the description below, the system 100 may also include an optical interface 112 for processing optical signals. More specifically, the catheter system 100 may include an optical detector disposed at the distal end 104 of the catheter 102. Such an optical detector detects optical signals transmitted through or reflected by the patient's blood. In either case, the optical signals are attenuated by the patient's blood in a manner which allows for calculation of oxygen saturation.

Similarly, the optical signals utilized by the oximeter may be transmitted by LEDs, for example, a red LED and an infrared LED, disposed at the distal end 104 of the catheter 102. In such a case, a drive signal for driving the sources is located in the oximeter instrument 114. The signals are electrically transmitted via the cable 108 to the sources at the distal end 104 of the catheter 102. The optical detector receives the optical signals and generates an electrical output signal representative of the received signals. The resulting detector signals are transmitted to the oximeter instrument 114 via the cable 108. It will be appreciated that, depending on the implementation, analog or digital signals may be used in this regard. Additionally, in certain applications, wireless signals may alternatively be used in this regard.

Alternatively, the sources and/or detector may be located at the oximeter instrument 114 or at some intermediate location between the oximeter instrument 114 and the catheter 102. In this regard, optical fibers may be used to couple the remotely located sources and/or detector to the distal end 104 of the catheter 102 where the oximetry measurements are desired. The illustrated system 100 includes an optical interface 112 in this regard. For example, the optical interface 112 may include appropriate optics to couple the proximate end of the optical fiber(s) to the sources and/or the detector. For example, each optical source may be optically coupled to a corresponding optical fiber using appropriate optical elements such as lenses or mirrors. Alternatively, multiple sources may be coupled to a single optical fiber by use of diffraction gradings, prisms, mirrors or the like, so as to provide a wavelength multiplexed signal. It will be appreciated that this signal may also be time division multiplexed, frequency division multiplexed or code division multiplexed. That is, each source is typically pulsed in a manner that allows for distinguishing between the contributions of each source to the detector signal and also for reducing noise.

As noted above, the sources and detector may be disposed at the distal end of the catheter or fiber optics may be utilized for transmitting optical signals to and from the distal end of the catheter. FIG. 2 illustrates a portion of a catheter 200 having sources and a detector disposed at a distal end 204 thereof. Specifically, the illustrated catheter includes a catheter body 202, a red LED 208, an infrared LED 210 and a photo detector 212. The catheter 200 further includes a tip electrode 206, such as an ablation electrode, and an electric wire cable 214 for electrically coupling the sources 208 and 210 and detector 212 to the oximeter instrument. Although not shown, electrical leads would be threaded through the catheter 200 to the tip electrode 206. It should be noted that, although an RF energy source is shown in FIG. 1, the ablation catheter 200 may use RF, cryo, microwave, ultrasonic or laser technologies. In addition, for certain applications, irrigation openings may be provided at the distal end 204 of the catheter 200 for irrigated procedures. In such cases, appropriate fluid channels are provided through the catheter to the openings.

In operation, drive signals transmitted via the cable 214 cause the sources 208 and 210 to flash according to a defined multiplexing scheme In this regard, the resulting optical signals may be time division multiplexed such that the sources 208 and 210 are alternately flashed, generally with a dark interval in between. Alternatively, pulse oximeters may be frequency division multiplexed or code division multiplexed.

In any event, the resulting optical signals are transmitted via a substantially transparent covering at the outside of the catheter 200 into the patient's blood. A portion of these optical signals is reflected back to the photo detector 212. The photo detector receives the incoming optical signals, generates an electrical signal representative of the received optical signals and transmits the electrical signal back to the oximeter instrument. Optionally, some signal processing and conditioning may be performed at the photo detector 212. For example, the signal may be converted from a current signal to a voltage signal, amplified, digitized or the like. Alternatively such signal processing may be performed at the oximetry instrument. Additional functionality such as separating the received signal into AC and DC components, de-multiplexing the signal, filtering, removing motion or other artifact and executing algorithms for calculating a value related to oxygen saturation may be performed by a processing unit, generally located at the oximeter instrument.

Alternatively, optical signals may be transmitted to and from the distal end of the catheter via optical fibers. A corresponding embodiment is shown in FIG. 3. The illustrated catheter 300 has a number of optical fiber ends 308, 310 and 312 disposed at a distal end 304 thereof. More specifically, the catheter 300 has a first fiber end 310 for transmitting the red and infrared optical signals. In the illustrated embodiment, two fiber ends 308 and 312 are used for detecting reflected optical signals. Specifically, fiber end 308 detects reflected light near to the transmitting fiber end 310 whereas the fiber end 312 detects reflected light farther from the transmitting fiber end 310. This arrangement is believed to allow for more accurate oxygen saturation readings and may also allow for improved monitoring as the interatrial septum is penetrated. In this regard, the signals from the fiber end 308 and the fiber end 312 may be combined or separately processed. The illustrated catheter further includes a catheter body 302, a tip electrode 306, as discussed above, and a fiber optical cable 314 for collecting the optical fibers.

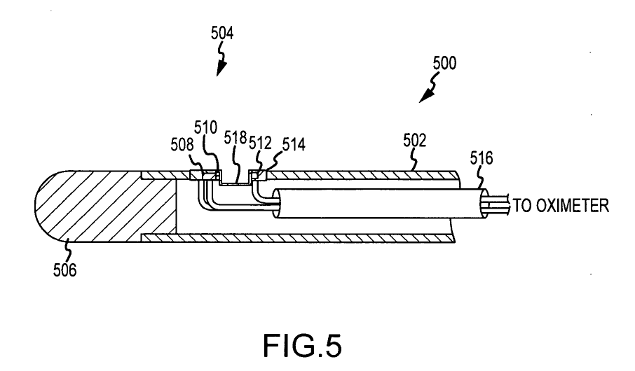

In the embodiments described above, the oximeter employed is a reflectance oximeter wherein the optical signals are transmitted into the patient's blood and reflected back to the detector or detector fiber ends. Alternatively, a transmittance-based oximeter may be employed where the optical signals are transmitted through the patient's blood to a detector disposed opposite the sources or source fiber ends. Such an embodiment is disclosed in FIG. 5. The illustrated catheter 500 has a recessed space 518 formed in a wall portion of the catheter at a distal end portion 504 thereof The red and infrared sources 508, 510 are disposed in opposing relationship to the photo detector 512 across the recessed space 518. In this manner, optical signals from the sources 508, 510 are transmitted through the patient's blood, which penetrates into the recessed space 518, so as to impinge directly on the photo detector 512. This may provide an improved signal in relation to reflectance-based oximetry arrangements. In the illustrated embodiment, the photo detector 512 is supportably encased within optically clear material 514. The illustrated catheter 500 further includes a catheter body 502, a tip electrode 506, as discussed above, and an electric wire cable 516 for collecting the electrical leads associated with the sources 508, 510 and the photo detector 512.

The recessed space 518 is dimensioned to accommodate placement of the sources 508, 510 and photo detector 512 on the sidewalls thereof as discussed above. In this regard, the recessed space 518 may have a depth, measured radially from an external wall of the catheter, of between about 0.010 and 0.040 inches. In addition, the recessed space 518 may have a width, measured along an axial dimension of the catheter 500, of between about 0.050 and 0.120 inches. The length of the recessed space 518, measured circumferentially in relation to the catheter 500, is sufficient to accommodate the sources 508, 510 and the photo detector 512. Optionally, the recessed space 518 may be covered by a sheath or the like as the catheter is introduced into the desired location for the procedure and withdrawn therefrom. It will be appreciated that optical fibers, as discussed above in connection with FIG. 3, may be used in connection with a recessed space/transmittance-based oximeter as disclosed in FIG. 5 with appropriate structural and dimensional modifications.

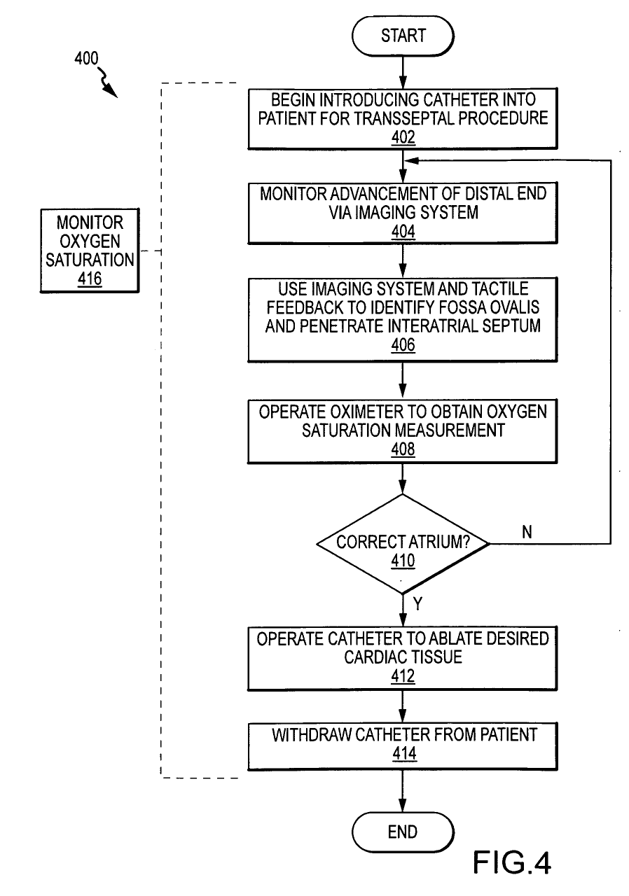

FIG. 4 is a flow chart illustrating a process 400 for performing a medical procedure in accordance with the present invention. The process 400 is initiated by beginning (402) introduction of the catheter into the patient for a transseptal procedure. For example, the catheter may be introduced into the patient's body through a vein or artery in the patient's leg, arm or neck. The catheter is then advanced through the patient's blood vessel to the patient's heart, for example, using a guide wire. Advancement of the distal end of the catheter may be monitored (404) via an imaging system. It is common to use fluoroscopic guidance and/or electrical signals together with previously acquired mapping information in this regard.

Once the distal end of the catheter has reached the patient's heart, the imaging system and tactile feedback can be used to identify (406) the fossa ovalis and to penetrate the interatrial septum. As noted above, the fossa ovalis is the thinnest portion of the interatrial septum and is generally the preferred location for penetrating the interatrial septum. Because this location is the thinnest part of the septum and generally the most compliant location, experienced physicians can identify this location via tactile feedback. The imaging system may also assist in this regard. In addition, some systems can assist in identifying the fossa ovalis based on electrical measurements of the tissue such as impedance measurements. In any event, once the physician is confident that the fossa ovalis has been identified, the distal end of the catheter is advanced to penetrate the interatrial septum.

As discussed above, successful penetration of the interatrial septum will be positively indicated by the oxygen saturation measurements from the oximetry structure at the distal end of the catheter. In this regard, the physician may view the current oxygen saturation measurements after penetration of the interatrial septum to verify proper positioning of the distal end of the catheter Alternatively, the physician may monitor the pulse oximeter readings during penetration of the interatrial septum to identify a change in oxygen saturation confirming penetration of the interatrial septum. As a still further alternative, as discussed above, such monitoring of oxygen saturation may be automated such that an indication can be provided to the physician upon penetration of the interatrial septum.

In this manner, the physician determines (410) whether the distal end of the catheter is in the correct atrium. If the distal end of the catheter is in the correct atrium, the physician can then operate (412) the catheter to ablate the desired cardiac tissue or otherwise perform a desired medical procedure. Otherwise, the physician continues to manipulate the catheter to attempt to attain the proper positioning. When the procedure is complete, the physician withdraws (414) the catheter from the patient. As shown, the oximetry measurements are not limited to positioning the distal end of the catheter in the correct atrium but may be monitored (416) throughout the procedure. For example, the oxygen saturation measurements may be monitored to provide an indication of patient health thereby eliminating the need for an external pulse oximeter. In addition, the oxygen saturation measurements may be monitored throughout the procedure as a further indication that the catheter is at the expected location, e.g., within a vein, artery or the like.

The foregoing description of the present invention has been presented for purposes of illustration and description. Furthermore, the description is not intended to limit the invention to the form disclosed herein. Consequently, variations and modifications commensurate with the above teachings, and skill and knowledge of the relevant art, are within the scope of the present invention. The embodiments described hereinabove are further intended to explain best modes known of practicing the invention and to enable others skilled in the art to utilize the invention in such or other embodiments and with various modifications required by the particular application(s) or use(s) of the present invention. It is intended that the appended claims be construed to include alternative embodiments to the extent permitted by the prior art.

What is claimed is:

1. A method for use in positioning a catheter at a desired location in a patient's body, comprising the steps of:
inserting said catheter into the patient's body;
performing at least one blood gas measurement in relation to said catheter with said catheter inserted into said patient's body; and
using said at least one blood gas measurement at least to assist in positioning said catheter at said desired location in the patient's body.

2. The method as set forth in claim 1, wherein said step of performing comprises measuring an oxygen saturation value or a carbon dioxide value.

3. The method as set forth in claim 1, wherein said step of performing comprises performing an optical measurement or a chemical measurement.

4. The method as set forth in claim 1, wherein said step of using comprises identifying a value of said at least one blood gas measurement indicating that said catheter is positioned at the desired location in the patient's body.

5. The method as set forth in claim 1, wherein said step of performing comprises performing a series of blood gas measurements and said step of using comprises monitoring said series of blood bas measurements during said step of inserting to identify a change in oxygen saturation related to positioning said catheter at the desired location in the patient's body.

6. The method as set forth in claim 1, wherein said step of performing comprises obtaining said at least one oxygen saturation measurement using oximetry structure disposed proximate to a distal end of said catheter.

7. The method as set forth in claim 1 wherein said step of using comprises using said at least one gas measurement to determine whether said distal end of said catheter is disposed in a right atrium or a left atrium of the patient's heart.

8. The method as set forth in claim 1, further comprising the step of operating an electrode disposed on said catheter to perform a medical procedure.

9. The method as set forth in claim 1, further comprising the step of operating an electrode disposed on said catheter to ablate cardiac tissue.

10. A catheter apparatus, comprising:
a therapeutic catheter having a distal end for introduction into a patient to a desired location via a blood vessel of the patient; and
blood gas measurement structure for measuring a blood gas related value proximate to said distal end of said catheter; and
medical procedure structure, disposed proximate to said distal end of said catheter and separate from said blood gas measurement structure, for performing a therapeutic medical procedure on said patient.

11. The apparatus as set forth in claim 10, wherein said blood gas measurement structure comprises optical structure, disposed proximate to said distal end of said catheter, for transmitting optical signals to said desired location of said patient and receiving said optical signals so as to permit measurement of said blood gas related value.

12. The apparatus as set forth in claim 11, wherein said optical structure includes transmission structure for transmitting a first optical signal at a first nominal wavelength and a second optical signal at a second nominal wavelength different from said first nominal wavelength, and receiving structure for receiving said first and second optical signals.

13. The apparatus as set forth in claim 12, wherein said transmission structure comprises at least one optical fiber end for transmitting said first and second optical signals.

14. The apparatus as set forth in claim 12, wherein said transmission structure comprises first and second LEDs for transmitting said first and second optical signals.

15. The apparatus as set forth in claim 12, wherein said transmission structure is operative for transmitting said first optical signal at a red wavelength and said second optical signal at an infrared wavelength.

16. The apparatus as set forth in claim 12, wherein said receiving structure comprises an optical fiber for receiving said first and second optical signals.

17. The apparatus as set forth in claim 12, wherein said receiving structure comprises a detector for receiving said first and second optical signals and providing first and second electrical signals representative thereof

18. The apparatus as set forth in claim 12, further comprising a processor for receiving information based on said optical signals and using said information to determine said oxygen saturation related value.

19. The apparatus as set forth in claim 12, wherein said medical procedure structure comprises at least one electrode disposed proximate to said distal end of said catheter.

20. The apparatus as set forth in claim 12, wherein said at least one electrode comprises an ablation electrode for ablating cardiac tissue.

21. The apparatus as set forth in claim 20, further comprising an ablation energy source operatively associated with said ablation electrode.

22. The apparatus as set forth in claim 21, further comprising instrumentation, operatively associated with said blood gas measurement structure, for processing a blood gas measurement input from said blood gas measurement structure so as to provide said blood gas related value.

23. The apparatus as set forth in claim 22, wherein said instrumentation comprises an optical interface for optically processing said gas measurement input to provide an electrical detector signal and an oximeter instrument for processing said electrical detector signal to provide said blood gas related value.

24. A method for use in performing catheter-based medical procedures on patients, comprising the steps of:
providing a catheter having blood gas measurement structure and medical procedure structure proximate to a distal end thereof;
introducing said distal end of said catheter into a body of a patient;
first operating said blood gas measurement structure to obtain at least one blood gas measurement; and
based at least in part on said at least one blood gas measurement, second operating said medical procedure structure to perform a medical procedure on said patient.

25. The method as set forth in claim 24, wherein said step of operating comprises transmitting a first optical signal of a first nominal wavelength and a second optical signal of a second nominal wavelength, different from said first nominal wavelength, into said patient and receiving said first and second optical signals after said signals have been attenuated by blood or perfused tissue of said patient.

26. The method as set forth in claim 24, wherein said step of introducing comprises inserting said distal end of said catheter into a heart of the patient.

27. The method as set forth in claim 24, wherein said step of introducing comprises inserting said distal end of said catheter through an interatrial septum of the patient.

28. The method as set forth in claim 24, wherein said step of second operating comprises ablating cardiac tissue of said patient.

29. The method as set forth in claim 24, wherein said step of first operating comprises using said blood gas measurement structure to monitor oxygen saturation measurements during insertion of said distal end of said catheter within the body of the patient.

30. The method as set forth in claim 29, further comprising the step of using said at least one blood gas measurement to position said distal end of said catheter at a desired location in said patient's body.
